(19) United States
(12) Patent Application Publication
(10) Pub. No.: US 2018/0350460 A1
(43) Pub. Date: Dec. 6, 2018

(54) IMAGE INTERPRETATION REPORT CREATION SUPPORT SYSTEM

(71) Applicant: OLYMPUS CORPORATION, Tokyo (JP)

(72) Inventors: Masaru HISANO, Tokyo (JP); Hirokazu NISHIMURA, Tokyo (JP)

(73) Assignee: OLYMPUS CORPORATION, Tokyo (JP)

(21) Appl. No.: 16/040,637

(22) Filed: Jul. 20, 2018

Related U.S. Application Data
(63) Continuation of application No. PCT/JP2016/080327, filed on Oct. 13, 2016.

(30) Foreign Application Priority Data
Jan. 21, 2016 (JP) 2016-009472

(51) Int. Cl. G16H 15/00; G16H 30/20; G16H 30/40; G06T 7/00

(52) U.S. Cl. CPC G16H 15/00; G16H 30/20; G06T 2207/10068; G06T 7/0012; G16H 30/40

(57) ABSTRACT

A first report input screen generation unit generates a first report input screen in which a first image interpreter inputs a result of image interpretation, and a second report input screen generation unit generates a second report input screen in which a second image interpreter inputs a result of image interpretation. A coordination processing unit causes input data input by the first image interpreter in the first report input screen to be reflected in an input area in the second report input screen. The first report input screen generation unit generates the first report input screen including a first comment input area in which the first image interpreter inputs a comment for the second image interpreter.

### CROSS-REFERENCE TO RELATED APPLICATIONS

This application is based upon and claims the benefit of priority from the prior Japanese Patent Applications No.2016-009472, filed on Jan. 21, 2016, and International Application No. PCT/JP2016/080327, filed on Oct. 13, 2016, the entire contents of which are incorporated herein by reference.

### BACKGROUND

The disclosure relates to a system for supporting the task of creating an image interpretation report.

Japanese Unexamined Patent Application Publication No.2014-67229 discloses a medical network system in which an image interpreting doctor in charge of checking can check and correct, as necessary, the content of an image interpretation report created by a trainee doctor or an inexperienced image interpreting doctor.

In a capsule endoscopic examination, the patient swallows a capsule having a built-in ultracompact camera from the mouth, with a plurality of antennas being fixed to the abdomen and a recorder being attached to the waist by a belt. The capsule captures still images periodically as it moves through the digestive tract and transfers captured images to the recorder from the antennas. After about 8 hours, the antennas and the recorder are collected, and the images recorded in the recorder are recorded in a database in medical facilities.

The image interpreter then observes images played back sequentially and extracts an image found to include a pathological abnormality. In a capsule endoscopic examination, several tens of thousands of images are captured so that the work load is known to be heavy. Double image interpretation by two image interpreting doctors is sometimes practiced to reduce the likelihood of overlooking. Recently, double image interpretation in which a qualified technician assists in diagnostic imaging and a doctor completes an image interpretation report is proposed.

In double image interpretation, the two image interpreters independently create an image interpretation report. In this process, it is preferable to support efficient creation of a report. In the case of double image interpretation performed by a combination of an technician and a doctor, development of a technology to support creation of an image interpretation report that also serves the purpose of educating the technician is called for. This is true not only of a combination of an technician and a doctor but also of a combination of an unskilled doctor and a skilled doctor. Development of a system capable of educating an unskilled image interpreter in the process of creating an image interpretation report is called for.

### SUMMARY

In this background, a purpose of the present disclosure is to provide an image interpretation report creation support technology to practice double image interpretation efficiently.

An image interpretation report creation support system for supporting a task of creating an image interpretation report according to an embodiment of the present disclosure is for: generating a first report input screen in which a first image interpreter inputs a result of image interpretation, the first report input screen not including a diagnostic input area in which to input a diagnostic result and including a first comment input area in which the first image interpreter inputs a comment for a second image interpreter; storing input data input by the first image interpreter in the first report input screen in a first storage unit; and performing a coordination process of reading out, when a second report input screen in which a second image interpreter inputs a result of image interpretation and which includes a diagnostic input area in which to input a diagnostic result is generated, the input data stored in the first storage unit and causing the input data to be reflected in an input area in the second report input screen.

Optional combinations of the aforementioned constituting elements, and implementations of the disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 shows a configuration of an image interpretation report creation support system according to an embodiment;

FIG. 2 shows a configuration of the report processing device;

FIG. 3 shows an example of a selection screen for selection of an endoscopic image;

FIG. 4 shows an example of a selected image display screen;

FIG. 5 shows an example of the first report input screen;

FIG. 6 shows an example of input in the first report input screen;

FIG. 7 shows an example of the second report input screen;

FIG. 8 shows an example of a selection screen for selection of an endoscopic image;

FIG. 9 shows an example of a selected image display screen;

FIG. 10 shows an example of the second report input screen;

FIG. 11 shows an example of input in the second report input screen;

FIG. 12 shows an example of the first report input screen; and

FIG. 13 shows a screen for setting items that should be reflected.

### DETAILED DESCRIPTION

One aspect of the disclosure will now be described by reference to the preferred embodiments. This does not intend to limit the scope of the present invention, but to exemplify the invention.

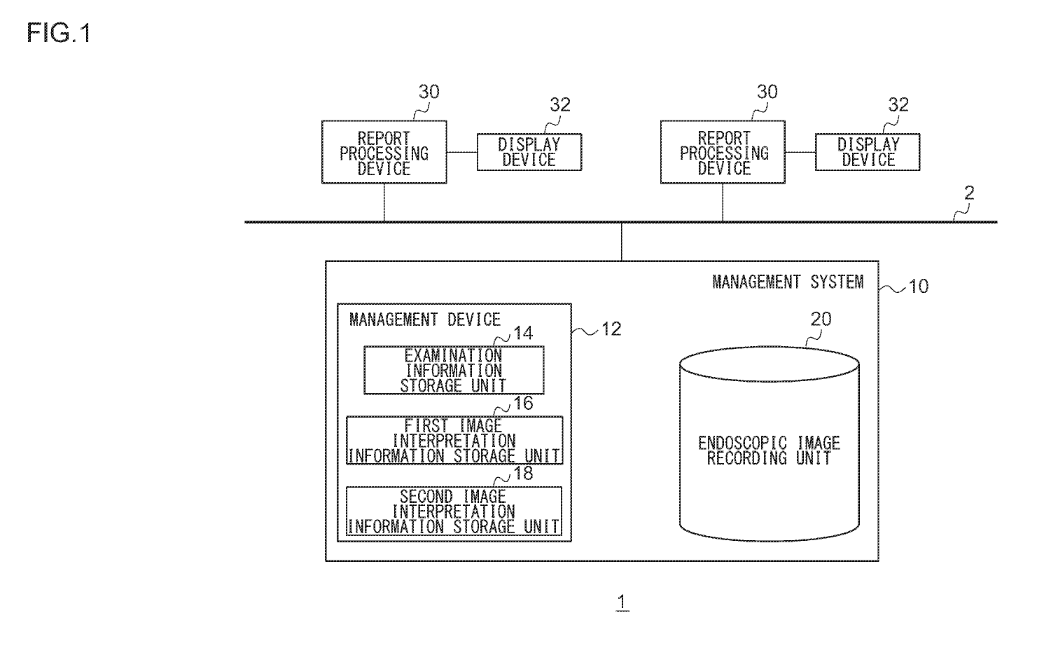

FIG. 1 shows a configuration of an image interpretation report creation support system 1 according to an embodiment of the present disclosure. The image interpretation report creation support system 1 is a system for supporting the task of creating an image interpretation report for a capsule endoscope and is provided with a management system 10 and a plurality of report processing devices 30. Each report processing device 30 is connected to the management system 10 via a network 2 such as a local area network (LAN). For example, the report processing device 30 is a terminal device such as a personal computer allocated to a doctor or an technician and is connected to a display device 32 to enable screen output. The report processing device 30 may be a laptop computer integrated with the display device or a mobile tablet. The report processing device 30 may be comprised of a terminal device and a server.

The management system 10 is provided with a management device 12 for managing order information for an endoscopic examination and image interpretation report information, and an endoscopic image recording unit 20 for recording endoscopic images captured by the capsule endoscope. The management device 12 is provided with an examination information storage unit 14 for storing order information for a patient, a first image interpretation information storage unit 16 and a second image interpretation information storage unit 18 for storing image interpretation report information input in the report processing device 30 from a report input screen.

The endoscopic image recording unit 20 records endoscopic images captured by the capsule endoscope. The endoscopic image recording unit 20 may be comprised of a hard disk drive (HDD) or a flash memory. Several tens of thousands of still images are captured in one session of capsule endoscopic examination. Therefore, the endoscopic image recording unit 20 needs to be configured as a large capacity database.

The report processing device 30 can access the management system 10 and display an endoscopic image recorded in the endoscopic image recording unit 20 on the display device 32. It is preferable that the management system 10 runs a playback application for endoscopic images and sequentially plays back several tens of thousands of endoscopic images efficiently. The playback application has a function of adjusting the playback speed in response to an instruction for playback from the report processing device 30 (e.g., an instruction for normal playback, an instruction for fast-forward playback, an instruction for fast-backward playback, etc.).

One of the purposes of a capsule endoscopic examination is to find a bleeding point in a digestive tract. For this purpose, the management system 10 identify, when the endoscopic images have been recorded in the endoscopic image recording unit 20, an endoscopic image considered to capture a bleeding state through image processing and marks the endoscopic image considered to include a bleeding state in advance. For example, the management system 10 identifies a reddish image as an endoscopic image considered to capture a bleeding state in advance. This allows an image interpreter to know an endoscopic image likely to include a bleeding state easily as the playback application plays back the images sequentially.

The report processing device 30 has a function of supporting creation of an image interpretation report by the image interpreter in coordination with the management system 10. A user interface such as a keyboard and a mouse is connected to the report processing device 30. The report processing device 30 causes the display device 32 to display an image interpretation report input screen. The image interpreter views the display screen and uses the user interface to input image interpretation information.

In creating an image interpretation report, the image interpreter first selects an endoscopic image found to include a pathological abnormality from several tens of thousands of endoscopic images captured. In the embodiment, double image interpretation by a combination of an technician and a doctor is performed. The technician and the doctor perform image interpretation respectively and create an image interpretation report. Generally, image interpretation means observing images and making a diagnosis. However, the role of the technician in double image interpretation according to the embodiment is the job of assisting in diagnostic imaging comprised of selecting an image including a pathological abnormality and inputting finding information and is not the act of diagnosis. In this sense, the technician is an image interpretation assistant who does not make a diagnosis, but the technician as well as the doctor will be referred to as an image interpreter hereinafter.

In the embodiment, the technician first assists in diagnostic imaging and creates an image interpretation report. Subsequently, the doctor creates an image interpretation report. The report processing device 30 according to the embodiment has a function of generating two types of report input screens with different formats. More specifically, the report processing device 30 has a function of generating a first report input screen for the technician to input a result of image interpretation and a second report input screen for the doctor to input a result of image interpretation. A representative difference between the two types of formats is that second report input screen is provided with an input field where the doctor inputs a diagnostic result, but the first report input screen is not provided with an input field for a diagnostic result.

To perform double image interpretation by the technician and the doctor efficiently, it is ensured that the doctor can check, in the second report input screen, the endoscopic image selected by the technician or the finding information input by the technician in the first report input screen. The report processing device 30 causes the data that the technician has input in the first report input screen to be reflected in an input area in the second report input screen for the doctor to create an image interpretation report. The doctor completes the image interpretation report by referring to the endoscopic image selected by the technician or the finding information input by the technician.

The detail of the report processing device 30 will be described below.

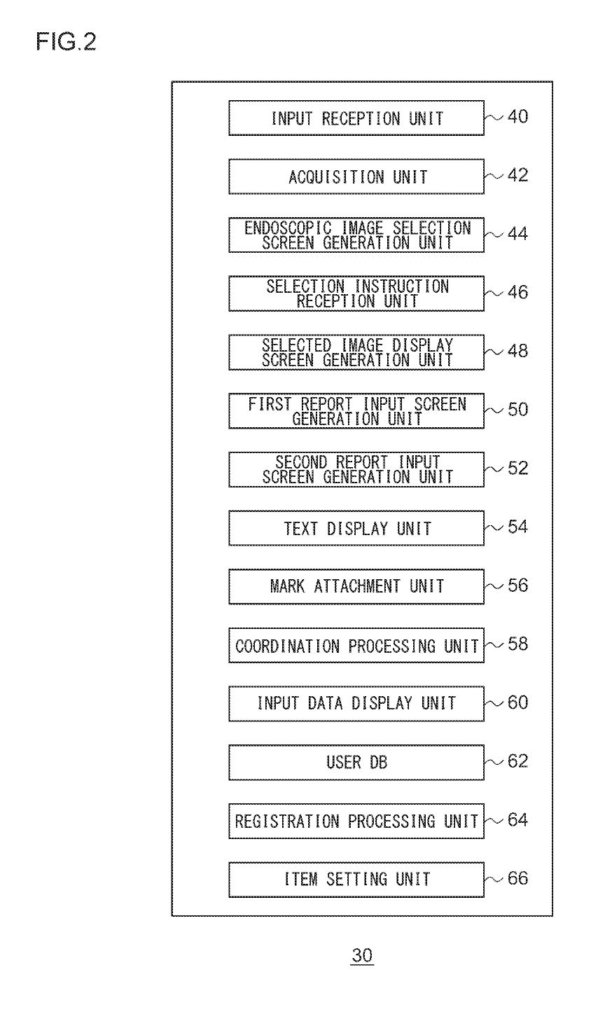

FIG. 2 shows a configuration of the report processing device 30. The report processing device 30 is comprised of an input reception unit 40, an acquisition unit 42, an endoscopic image selection screen generation unit 44, a selection instruction reception unit 46, a selected image display screen generation unit 48, a first report input screen generation unit 50, a second report input screen generation unit 52, a text display unit 54, a mark attachment unit 56, a coordination processing unit 58, an input data display unit 60, a user database (DB) 62, a registration processing unit 64, and an item setting unit 66. The input reception unit 40 receives a user operation input by the doctor in a user interface such as a mouse and a keyboard.

The features are implemented in hardware such as an arbitrary processor, a memory, or other LSI's and in software such as a program loaded into a memory. The figure depicts functional blocks implemented by the cooperation of these elements. Therefore, it will be understood by those skilled in the art that the functional blocks may be implemented in a variety of manners by hardware only, software only, or by a combination of hardware and software. As described above, the report processing device 30 may be a terminal device or comprised of a terminal device and a server. Therefore, the functions shown in FIG. 2 as the features of the report processing device 30 may be implemented by a device other than the terminal device.

An example of practicing double image interpretation by an technician B and a doctor C will be described below. The technician B enters the user ID to log into the report processing device 30. The user DB 62 stores the user ID and the user attribute information, mapping them to each other. The user attribute information includes information indicating whether the user is an technician or a doctor. When the user ID entered is received, the input reception unit 40 refers to the user DB 62 and identifies that the user is an technician. When the user logging in is an technician, the report input screen is generated by the first report input screen generation unit 50. When the user logging in is a doctor, the report input screen will be generated by the second report input screen generation unit 52.

When the technician B logs in, the management device 12 supplies examination information stored in the examination information storage unit 14 to the report processing device 30, and the display device 32 displays a list of capsule endoscopic examinations. The list of examinations lists examination information such as the patient ID, patient name, examination ID, date and time of examination, and the technician B selects an examination in which the technician B is expected to create an image interpretation report. When an examination for a patient A and with an examination ID “0001” is selected from the list of examinations, the input reception unit 40 identifies the examination ID in response to an input for selection of the examination. The endoscopic image selection screen generation unit 44 generates a selection screen for selecting an endoscopic image and causes the display device 32 to display the selection screen.

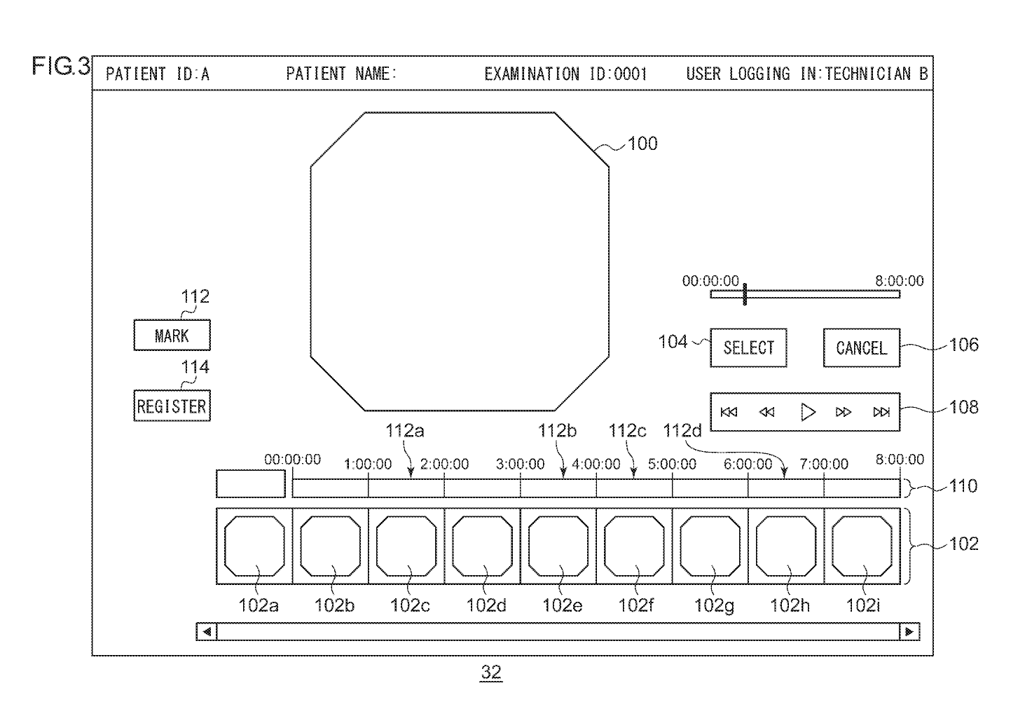

FIG. 3 shows an example of a selection screen for selection of an endoscopic image. The acquisition unit 42 acquires endoscopic images stored in the endoscopic image recording unit 20 for sequential playback from the playback application of the management system 10. The endoscopic image selection screen generation unit 44 outputs images for sequential play back to the playback area 100. The management system 10 processes endoscopic images to allow efficient sequential playback. For example, similar images (images with small image-to-image variations) are pre-processed for high-speed playback. When the user uses a selection button 104 while images are being sequentially played back in the playback area 100, the endoscopic image displayed in the playback area 100 is captured and displayed in a selected image display area 102.

When an image found to include a pathological abnormality is displayed in the playback area 100, the technician B uses the selection button 104 to select the displayed image. When the selection instruction reception unit 46 receives a user operation in the selection button 104 as an instruction to select the image and transmits the instruction to the management system 10, the management system 10 extracts the image displayed when the instruction for selection is received as the selected image. The management system 10 transmits the selected image thus extracted to the report processing device 30, and the selected image is displayed in the selected image display area 102. The image displayed in the selected image display area 102 may be selected as an image attached to an image interpretation report later.

Of pathological abnormalities, the technician B must select the image capturing a bleeding state without exception. When the management system 10 has identified an endoscopic image considered to capture a bleeding state in advance through image processing, information indicating the fact may be displayed in the playback area 100 or near the playback area 100 when the image considered to capture the bleeding state is displayed in the playback area 100. This allows the technician B to recognize that the image likely to include a bleeding state is being displayed. By causing the management system 10 to identify an endoscopic image considered to capture a bleeding state in advance through image processing, the risk of overlooking by the image interpreter can be reduced.

FIG. 3 shows that selected images 102a˜102i are selected in the selected image display area 102. The user may select the selected image in the selected image display area 102 and use a cancel button 106 to cancel the selection. For example, the cancel button 106 may be used to maintain only a properly captured image if a plurality of images are selected in connection the same finding. Buttons 108 for controlling the playback operation are for controlling the playback operation in the playback area 100 and enable adjustment of the playback speed.

A timeline 110 in the selection screen for selection of an endoscopic image is a user interface to indicate the temporal position of the endoscopic image played back in the playback area 100 and is also a user interface to display an endoscopic image. When the technician B places the mouse pointer at a desired section on the timeline 110, the endoscopic image captured at that point of time is displayed in the playback area 100. The technician B can add a mark in the timeline 110 to indicate a position where a body part starts. A mark 112a indicates the start of the stomach, a mark 112b indicates the start of the duodenum, a mark 112c indicates the start of the jejunum, and a mark 112d indicates the start of the colon. The technician B viewing endoscopic images played back sequentially in the playback area 100 and finding that a new body part is played back uses a marking button 112 to mark the position of start of the body part on the timeline. In this way, the technician B performs image observation by marking the positions of start of body parts and selecting the endoscopic image capturing a pathological abnormality. Marking makes it easy to know where to start when reviewing endoscopic images later.

The technician B having finished observing the images uses a registration button 114. When registration button 114 is used, the selected image selected in the selected image display area 102 is stored in the first image interpretation information storage unit 16 and the endoscopic image recording unit 20 in the management system 10, and time information indicating the positions of start of the body parts marked by using the marking button 112 are stored in the endoscopic image recording unit 20. When the task of image observation is completed, the technician B subsequently creates an image interpretation report. The technician B starts a report creation application for creating an image interpretation report in the report processing device 30.

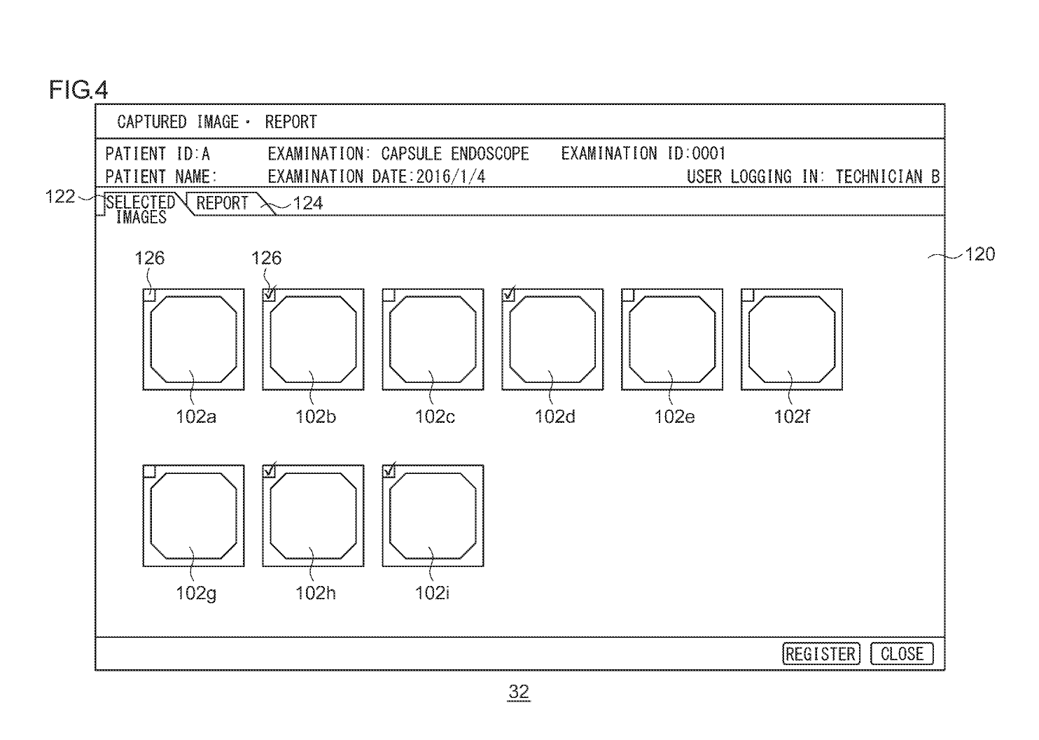

FIG. 4 shows an example of a selected image display screen generated by the report creation application. When the report creation application is started, the acquisition unit 42 acquires the thumbnail of the selected image linked to the examination ID from the first image interpretation information storage unit 16. The selected image stored in the first image interpretation information storage unit 16 is the image selected in the endoscopic image selection screen shown in FIG. 3.

The selected image display screen generation unit 48 displays a list of thumbnails of the selected endoscopic images in a thumbnail display area 120. When the first image interpretation information storage unit 16 does not store the thumbnail, the acquisition unit 42 may acquire the selected image and the selected image display screen generation unit 48 may reduce the selected image to generate a thumbnail and display the thumbnail in the thumbnail display area 120. The selected image display screen is displayed on the display device 32 such that a selected image tab 122 is selected.

A checkbox 126 is provided for each thumbnail. When the technician B locates the mouse pointer in the checkbox 126 and clicks the left mouse button, the thumbnail is selected as an image attached to the image interpretation report. The technician B can enlarge the thumbnail from a menu displayed by locating the mouse pointer on the thumbnail and clicking the right mouse button. The technician B may determine whether to attach the image to the report by viewing the enlarged endoscopic image. FIG. 4 shows that selected images 102b, 102d, 102h, and 102i are selected as images attached to the report.

When the technician B selects the attached image in the selected image display screen and then selects a report tab 124, the first report input screen generation unit 50 causes the display device 32 to display the first report input screen.

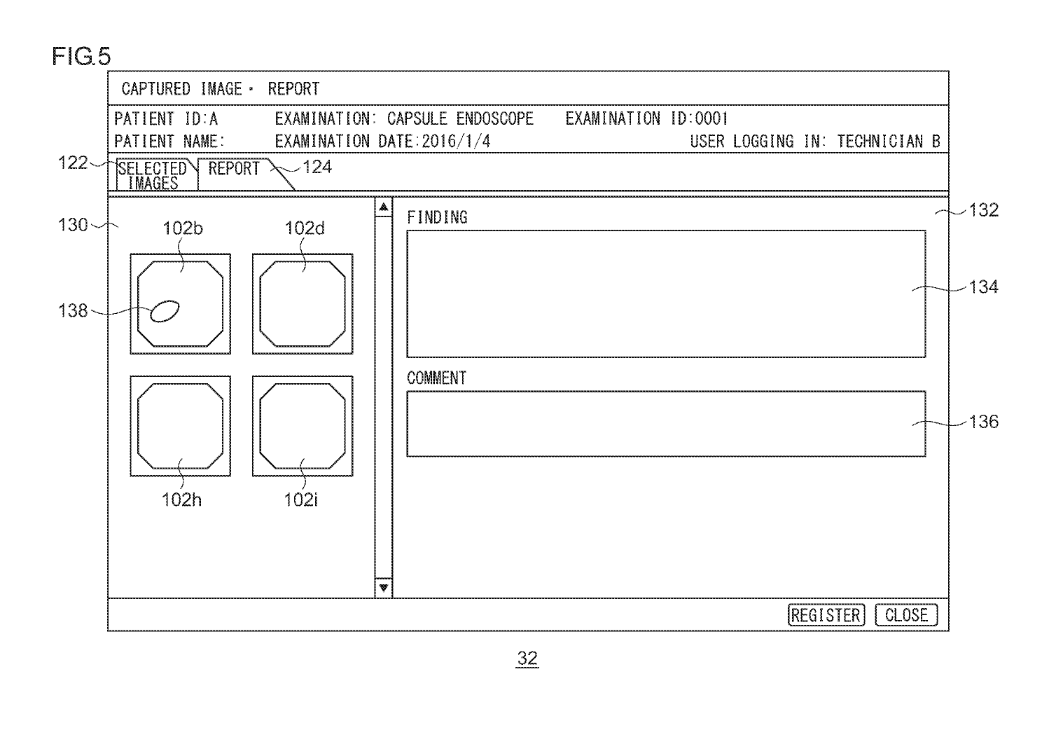

FIG. 5 shows an example of the first report input screen. The first report input screen generation unit 50 generates the first report input screen in which the technician (the first image interpreter) inputs the result of image interpretation and causes the display device 32 to display the first report input screen. The first report input screen includes two areas. On the left is located an attached image display area 130 to display a thumbnail of the attached image. On the right is located an image interpretation result input area 132 in which the image interpreter inputs the result of image interpretation.

The first report input screen generation unit 50 locates the image selected in the thumbnail display area 120 shown in FIG. 4 in the attached image display area 130. In this case, the selected images 102b, 102d, 102h, and 102i selected as the images attached to the report are displayed in the attached image display area 130.

The image interpretation result input area 132 is an area in which the technician writes the result of image interpretation by using a keyboard. The image interpretation result input area 132 includes a finding input area 134 in which the technician inputs finding information, and a comment input area 136 in which the technician inputs a comment for the doctor.

The technician B inputs finding information in the finding input area 134. The technician B may also add predetermined information to the selected image displayed in the attached image display area 130. In this case, the figure shows that an attention mark 138 is attached to selected image 102b. When the technician B performs a predetermined mouse operation over the selected image, a mark attachment unit 56 attaches the attention mark 138 indicating a bleeding point to the selected image. In association with this, the technician B writes, in the finding input area 134, that bleeding is identified in the selected image 102b as a finding detail.

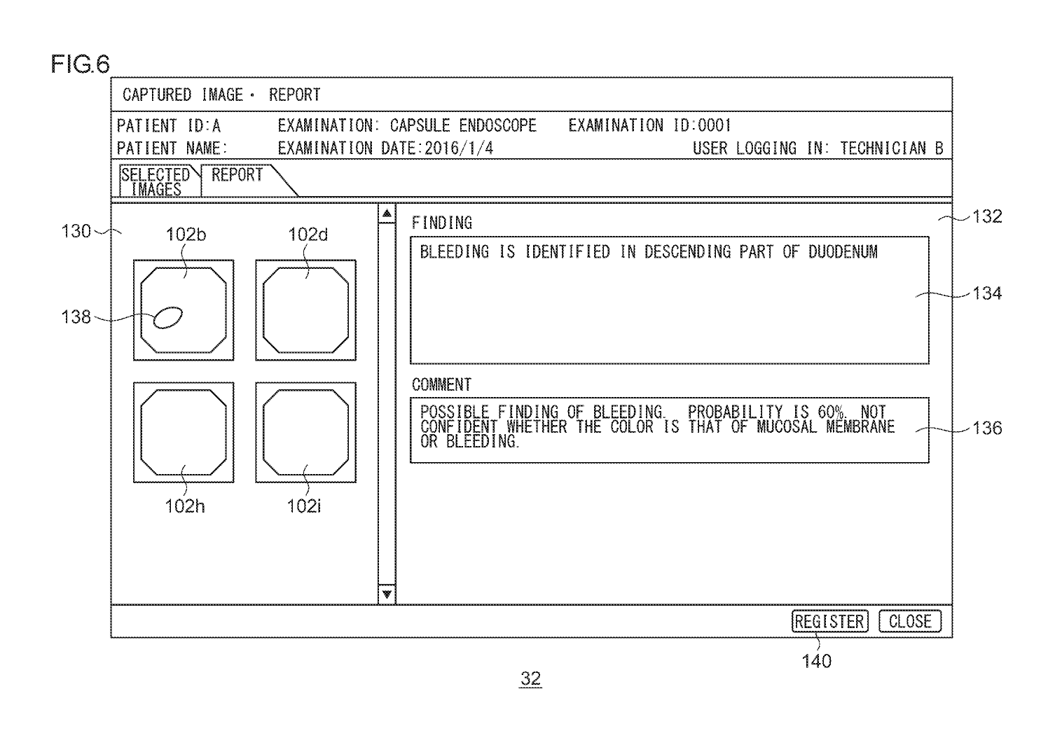

FIG. 6 shows an example of input in the first report input screen. The technician B inputs finding information in the finding input area 134. In this case, the finding information “Bleeding is identified in the descending part of the duodenum” is input by using a keyboard. The input reception unit 40 receives characters input via the keyboard and the text display unit 54 displays the characters in the finding input area 134. The technician B also inputs a comment for the doctor C in the comment input area 136. The technician B may write a question regarding the input finding, or confidence or a lack thereof in the comment input area 136 in a free format. The entry in the comment input area 136 is later checked by the doctor C. The doctor C can know how the technician B judged the finding information.

When the technician B has input the result of image interpretation and uses a registration button 140, the registration processing unit 64 registers the data input by the technician B in the first report input screen in the first image interpretation information storage unit 16. The input data registered includes the input data in the finding input area 134 and the comment input area 136, the image selected in the attached image display area 130 as the attached image, and the information related to the attention mark 138 attached to the image. In this way, the first image interpretation information storage unit 16 stores the input data input by the technician B in the first report input screen.

In this embodiment, the input data input by the technician B in the first report input screen is reflected in the input area of the second report input screen in which the doctor C inputs the result of image interpretation. Before describing the process for reflection, a description will be given of a configuration of the second report input screen.

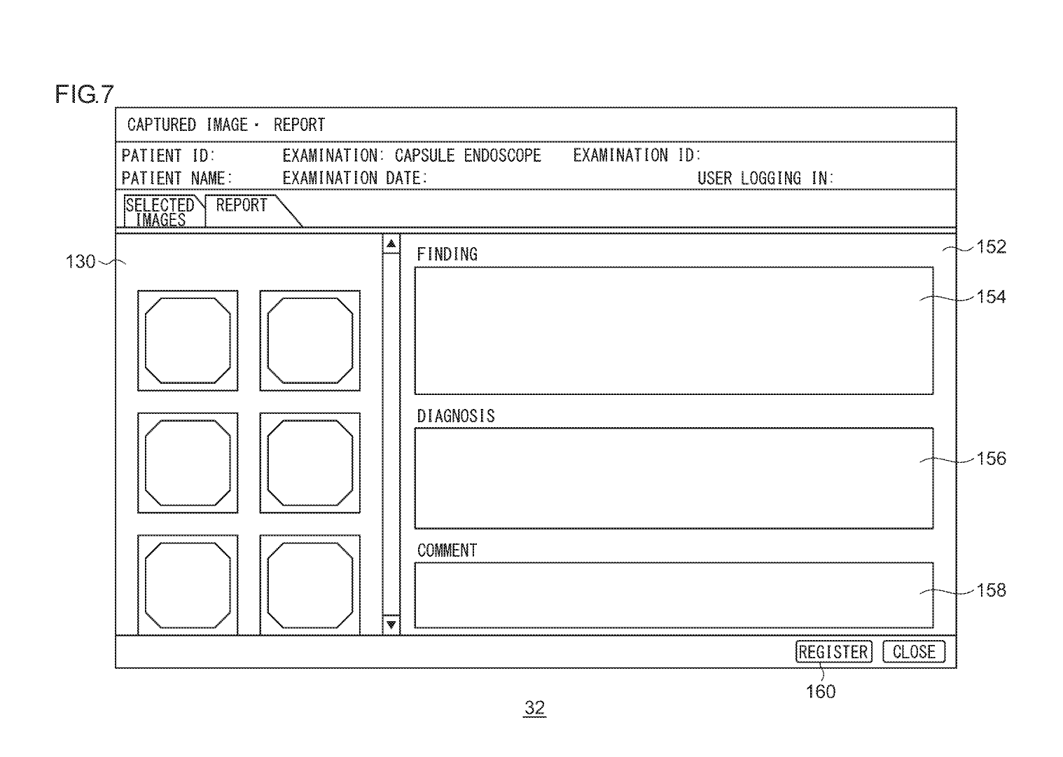

FIG. 7 shows an example of the second report input screen. The second report input screen generation unit 52 generates the second report input screen in which the doctor (the second image interpreter) inputs the result of image interpretation and causes the display device 32 to display the second report input screen. The second report input screen includes two areas. On the left is located an attached image display area 130 to display a thumbnail of the attached image. On the right is located an image interpretation result input area 152 in which the image interpreter inputs the result of image interpretation. The second report input screen generation unit 52 locates the selected image in the attached image display area 130.

The image interpretation result input area 152 is an area in which the doctor writes the result of image interpretation by using a keyboard. The image interpretation result input area 152 includes a finding input area 154 in which the doctor inputs finding information, a diagnostic input area 156 in which the doctor inputs diagnostic information, and a comment input area 158 in which the doctor inputs a comment for the technician. When the doctor has input the result of image interpretation and uses a registration button 160, the registration processing unit 64 registers the data input by the doctor in the second report input screen in the second image interpretation information storage unit 18. Given above is a general explanation of the second report input screen.

In the embodiment, the doctor C can check, in the second report input screen, the result of image interpretation input by the technician B. For this purpose, the report processing device 30 performs the process of deploying the data input by the technician B on the second report input screen as the doctor C opens the second report input screen.

A description will now be given of image interpretation by the doctor C. The doctor C enters the user ID to log into the report processing device 30. When the user ID entered is received, the input reception unit 40 refers to the user DB 62 and identifies that the user is a doctor. When the user logging in is a doctor, the report input screen is generated by the second report input screen generation unit 52.

When the doctor C logs in, the management device 12 supplies examination information stored in the examination information storage unit 14 to the report processing device 30, and the display device 32 displays a list of capsule endoscopic examinations. When the doctor C selects an examination for a patient A and with an examination ID “0001” from the list of examinations, the input reception unit 40 identifies the examination ID in response to an input for selection of the examination. The endoscopic image selection screen generation unit 44 generates a selection screen for selecting an endoscopic image and causes the display device 32 to display the selection screen.

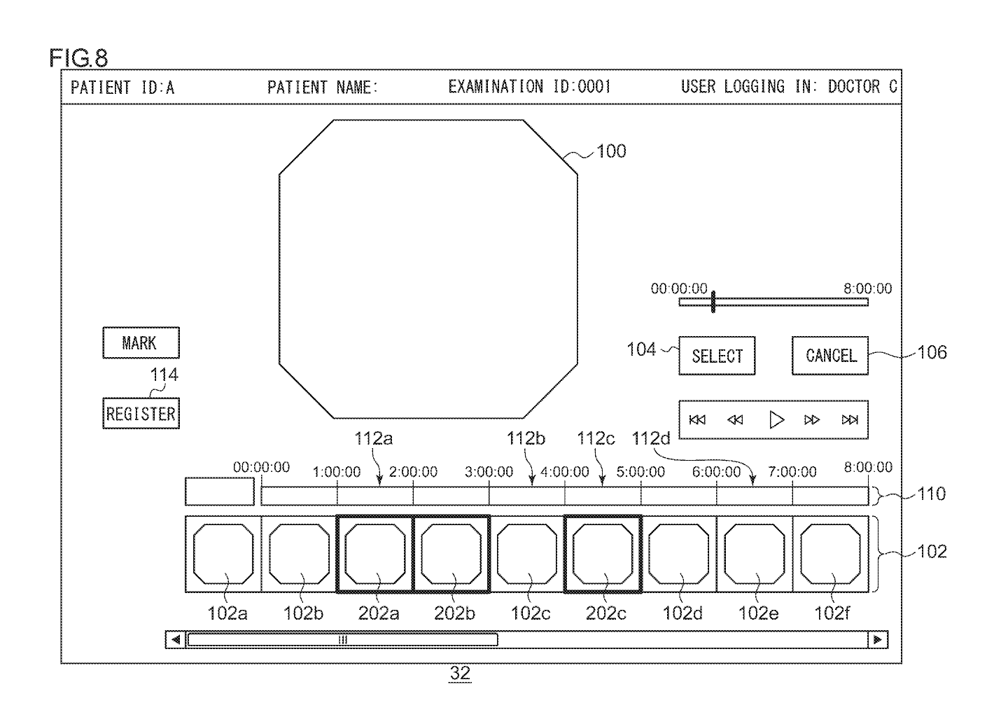

FIG. 8 shows an example of a selection screen for selection of an endoscopic image. The acquisition unit 42 acquires endoscopic images stored in the endoscopic image recording unit 20 for sequential playback from the playback application of the management system 10. The endoscopic image selection screen generation unit 44 outputs images sequentially played back to the playback area 100. In this process, the coordination processing unit 58 acquires the selected images stored in the endoscopic image recording unit 20 as a result of the task of observation performed by the technician B and the time information indicating the positions of start of the respective body parts. The endoscopic image selection screen generation unit 44 displays the marks 112a˜112d and the selected images 102a˜102i. The doctor C can know the positions of start of the respective body parts by referring to the marks 112a˜112d placed by the technician B. FIG. 8 shows that images 202a˜202c selected by the doctor C are additionally displayed in the selected image display area 102 and the selected images 102g˜102i are not displayed. By moving the scroll bar at the bottom, the selected images 102g˜102i are displayed in the selected image display area 102.

The doctor C observers the images displayed in the playback area 100, and, when an image found to include a pathological abnormality is displayed in the playback area 100, the doctor C uses the selection button 104 to select the displayed image. When the doctor C uses the selection button 104, the selection instruction reception unit 46 receives a user operation in the selection button 104 as an instruction to select the image and transmits the instruction to the management system 10. The management system 10 extracts the displayed image occurring when the instruction for selection is received as the selected image. The management system 10 transmits the selected image thus extracted to the report processing device 30, and the selected image is displayed in the selected image display area 102. The task of observation described above is the same as the task of observation described in connection with the technician B.

The doctor C can refer to the selected images 102a˜102i selected by the technician B and so can surmise the intent with which the technician B selected the image. The doctor C need not select the same image as selected by the technician B. When the technician B has selected an image found to include a pathological abnormality and when a further image captures the pathological abnormality more clearly, the doctor C selects the further image. The doctor C may de-select the unclear image selected by the technician B by using the cancel button 106.

FIG. 8 shows that the selected images 202a˜202c selected by the doctor C are displayed in addition to the selected images 102a˜102f selected by the technician B in the selected image display area 102. The endoscopic image selection screen generation unit 44 displays the selected images selected by the technician B and the selected images selected by the doctor C in the selected image display area 102 in a manner that the selected images selected by the technician B and the selected images selected by the doctor C can be distinguished. For example, the selected images 202a˜202c selected by the doctor C may be shown in a mode different from that of the selected images 102a˜102f selected by the technician B, by encircling the images with a bold frame. The display mode may not be limited to this. For example, the name of the image interpreter selecting the image may be displayed below each selected image. At any rate, the images may be displayed in a mode capable of distinguishing the images in terms of the selector.

The doctor C having finished observing the images uses the registration button 114. When registration button 114 is used, the selected image selected in the selected image display area 102 is stored in the second image interpretation information storage unit 18 and the endoscopic image recording unit 20 in the management system 10. When the task of image observation is completed, the doctor C subsequently creates an image interpretation report. The doctor C starts a report creation application for creating an image interpretation report in the report processing device 30.

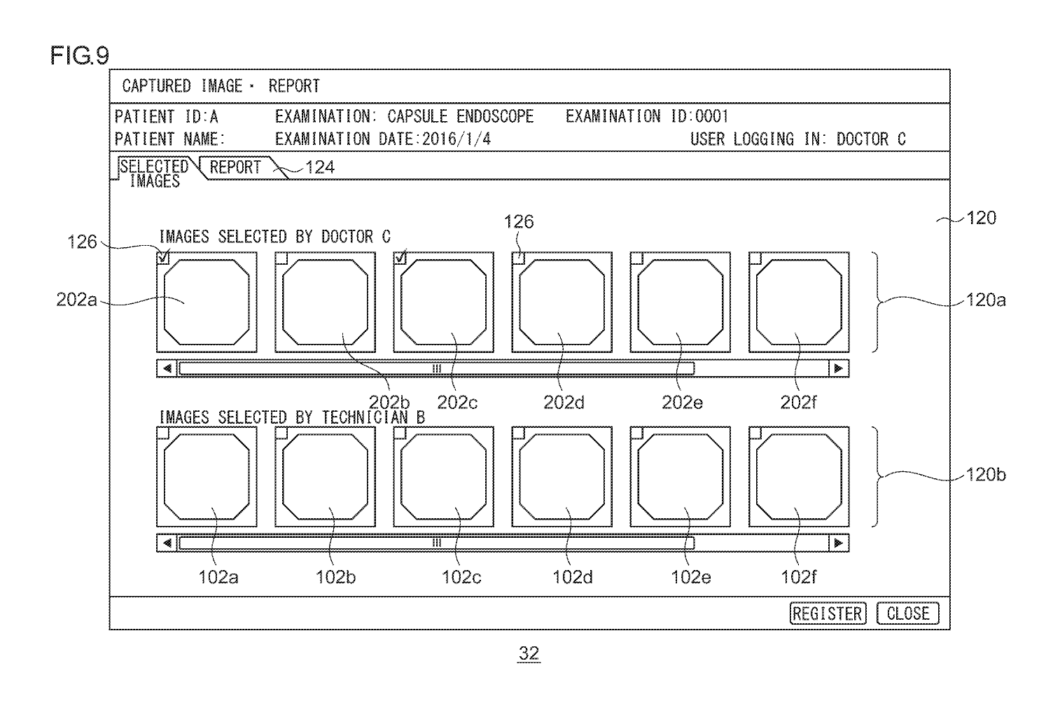

FIG. 9 shows an example of a selected image display screen generated by the report creation application. When the report creation application is started, the acquisition unit 42 acquires the thumbnail of the selected image linked to the examination ID from the second image interpretation information storage unit 18. The selected images stored in the second image interpretation information storage unit 18 include the images selected by the doctor C in the endoscopic image selection screen shown in FIG. 8 and further includes the images selected by the technician B.

The selected image display screen generation unit 48 displays the images selected by the doctor C and the images selected by the technician B in the thumbnail display area 120, distinguishing the images selected by the doctor C from the images selected by the technician B. A doctor-selected area 120a is a display area for the images selected by the doctor C and an technician-selected area 120b is a display area for the images selected by the technician B. The doctor C can have only those images attached to the report displayed in the selected image display screen shown in FIG. 9 by selecting an image attached to the report in the endoscopic image selection screen shown in FIG. 8 and canceling the selection of the images not attached. In the illustrated example, the doctor C does not cancel the selected images, and the images 102a˜102i selected by the technician B can be referred to.

When the doctor C locates the mouse pointer in the checkbox 126 and clicks the left mouse button, the thumbnail is selected as an image attached to the image interpretation report. In this case, the doctor C selects the selected images 202a, 202c as images attached to the report. The selected images 102b, 102d, 102h, 102i are selected by the technician B as images attached to the report.

A check mark may be entered in the check boxes for these selected images along with information indicating that the technician B has checked. When the doctor C selects the attached image in the selected image display screen and then selects the report tab 124, the second report input screen generation unit 52 causes the display device 32 to display the second report input screen.

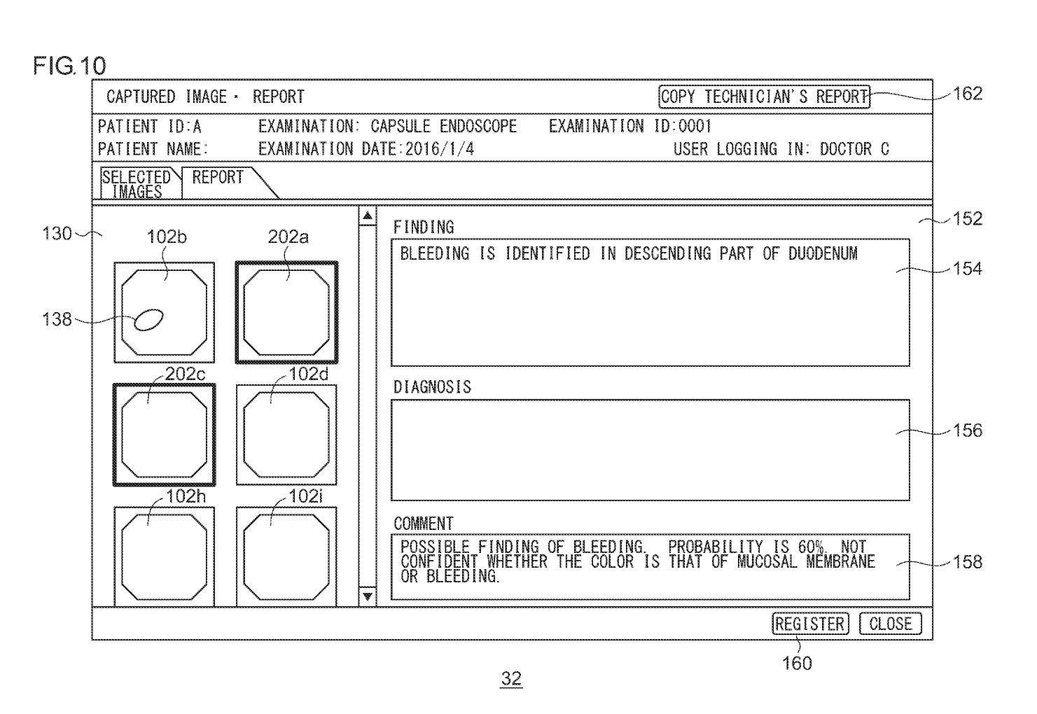

FIG. 10 shows an example of the second report input screen. The second report input screen generation unit 52 generates the second report input screen in which the doctor (the second image interpreter) inputs the result of image interpretation and causes the display device 32 to display the second report input screen. The second report input screen generation unit 52 locates the image selected in the thumbnail display area 120 shown in FIG. 9 in the attached image display area 130. In this case, the selected images 202a, 202c selected as the images attached to the report are displayed in the attached image display area 130.

In this process, the coordination processing unit 58 performs the process of reading the input data stored in the first image interpretation information storage unit 16 and causes the input data to be reflected in the input area in the second report input screen. The coordination processing unit 58 may determine whether the input data is stored in the first image interpretation information storage unit 16 for the same examination data. If the input data is stored, the coordination processing unit 58 may display a copy button 162 in the second report input screen. The copy button 162 is a user operation button for causing the input data entered by the technician B to be reflected in the input area in the second report input screen. When the doctor C uses the copy button 162, the coordination processing unit 58 may perform the process for reflection.

The first image interpretation information storage unit 16 stores, as the input data entered by the technician B, the input data in the finding input area 134 and the comment input area 136, the image selected in the attached image display area 130 as the attached image, and the information related to the attention mark 138 attached to the image. The coordination processing unit 58 reads these items of input data and causes them to be reflected in the input area in the second report input screen.

More specifically, the coordination processing unit 58 reads the selected images 102b, 102d, 102h, 102i selected by the technician b as the attached images from the first image interpretation information storage unit 16 and locates the selected images thus read in the attached image display area 130. The coordination processing unit 58 also reads the input data in the finding input area 134 from the first image interpretation information storage unit 16 and displays the input data in the finding input area 154. The coordination processing unit 58 also reads the input data in the comment input area 136 from the first image interpretation information storage unit 16 and displays the input data in the comment input area 158. The coordination processing unit 58 also reads the information related to the attention mark 138 attached to the selected image 102b from the first image interpretation information storage unit 16 and attaches the mark to the selected image 102b. This process is performed by the input data display unit 60. The input data display unit 60 performs the process of displaying the input data read by the coordination processing unit 58 in the input area designated by the coordination processing unit 58. This allows the doctor C to view, in the second report input screen, the input data in the image interpretation report created by the technician B.

In this process, it is preferable that the coordination processing unit 58 display the input data read from the first image interpretation information storage unit 16 in the input area in the second report input screen in a manner that it is known that the input data was entered by the technician. For example, the coordination processing unit 58 may set the character color of the text data entered by the technician B to be different from the character color of the text data entered by the doctor C. For example, given that the character color of the text data entered by the doctor C is black, the character color of the text data entered by the technician B may be set to red. This allows the doctor C to distinguish between characters entered by the doctor C and those entered by the technician B. What is required is that the doctor C can know that characters were entered by the technician B, and the coordination processing unit 58 may use different character sizes or different character fonts as well as different character colors. The name of the image interpreter may be displayed.

Further, the coordination processing unit 58 displays the attached images selected by the technician B and the attached images selected by the doctor C in the attached image display area 130 in different modes. In the illustrated examples, the attached images selected by the doctor C are encircled by a bold frame. Alternatively, the name of the image interpreter may be attached to each selected image. The doctor C can delete unnecessary attached images in the attached image display area 130.

The doctor C enters finding information in the finding input area 154. If the finding information entered by the technician B in the finding input area 154 is accurate, the doctor C need not enter finding information a second time. By causing the finding information entered by the technician B to be reflected in the finding input area 154, the doctor C can save the trouble of entry, while also ensuring that double image interpretation is performed. If the finding information entered by the technician B is deficient, the doctor C adds finding information. If the finding information entered by the technician B is in error, the doctor C deletes the erroneous finding information from the finding input area 154 and enters accurate finding information. In this way, the doctor C establishes the finding information in the finding input area 154.

In entering finding information, the doctor C refers to the comment from the technician B displayed in the comment input area 158. The doctor C may read the comment by the technician B to see how the technician B′s judgment was clouded. Upon reading the image interpretation report by the technician B, the doctor C enters a comment for the technician B in the comment input area 158. The doctor C may answer a question from the technician B or enter what the doctor C has noticed as a comment. The detail of the entry by the doctor C in the comment input area 158 is later checked by the technician B and will help improve the image interpretation skill of the technician B.

The doctor C also enters diagnostic information in the diagnostic input area 156. As described above, the diagnostic input area 156 is provided in the second report input screen in which the doctor enters data, but is not provided in the first report input screen in which the technician enters data.

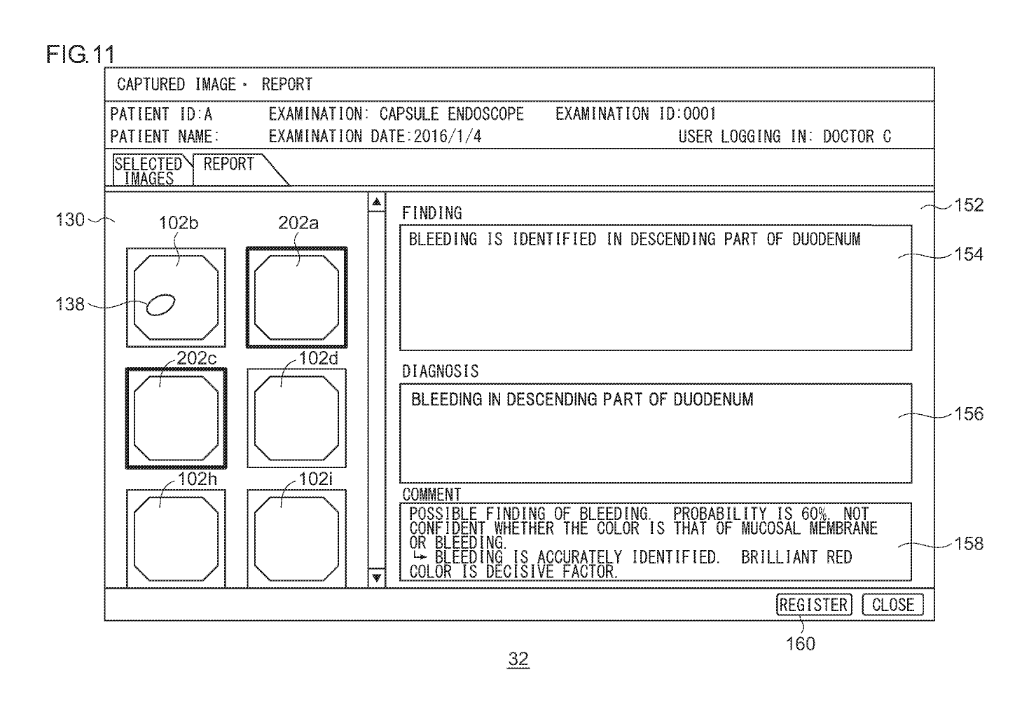

FIG. 11 shows an example of input in the second report input screen. Referring to the finding input area 154, the doctor C is using the input data entered by the technician B as it is and is not modifying the input data. Referring to the attached image display area 130, the attached images 102b, 102d, 102h, 102i selected by the technician B are also used as images attached to the ultimate report. The doctor C enters diagnostic information in the diagnostic input area 156 and enters an advice for the technician B in the comment input area 158.

When the doctor C has input the result of image interpretation and uses the registration button 160, the registration processing unit 64 registers the data input by the doctor C in the second report input screen in the second image interpretation information storage unit 18. The input data includes the input data in the finding input area 154, the diagnostic input area 156, and the comment input area 158, the image selected in the attached image display area 130 as the attached image, and the information related to the attention mark 138 attached to the image. In this way, the second image interpretation information storage unit 18 stores the input data input by the doctor C in the second report input screen. This completes the image interpretation by the doctor C.

The second image interpretation information storage unit 18 stores the input data input in the input area in the second report input screen. As shown in FIG. 11, the input data stored by the second image interpretation information storage unit 18 includes the data entered by the technician B in the first report input screen, i.e., the input data stored in the first image interpretation information storage unit 16 and caused by the coordination processing unit 58 to be reflected in the input area in the second report input screen. Thus, in the embodiment, the load on the doctor C in creating the report is reduced by exploiting the data in the input area in the second input screen, in which the input data entered by the technician B is reflected by the coordination processing unit 58, as the image interpretation information for the report by the doctor C.

The input data stored in the second image interpretation information storage unit 18 is used in printing the image interpretation report, but the input data in the comment input area 158 is not used in printing. The comment input area 136 in the first report input screen and the comment input area 158 in the second report input screen in the embodiment are solely used as a means of communication between the technician B and the doctor C. By providing the comment input areas, the doctor C can educate the technician B concurrently with creating the doctor's own image interpretation report.

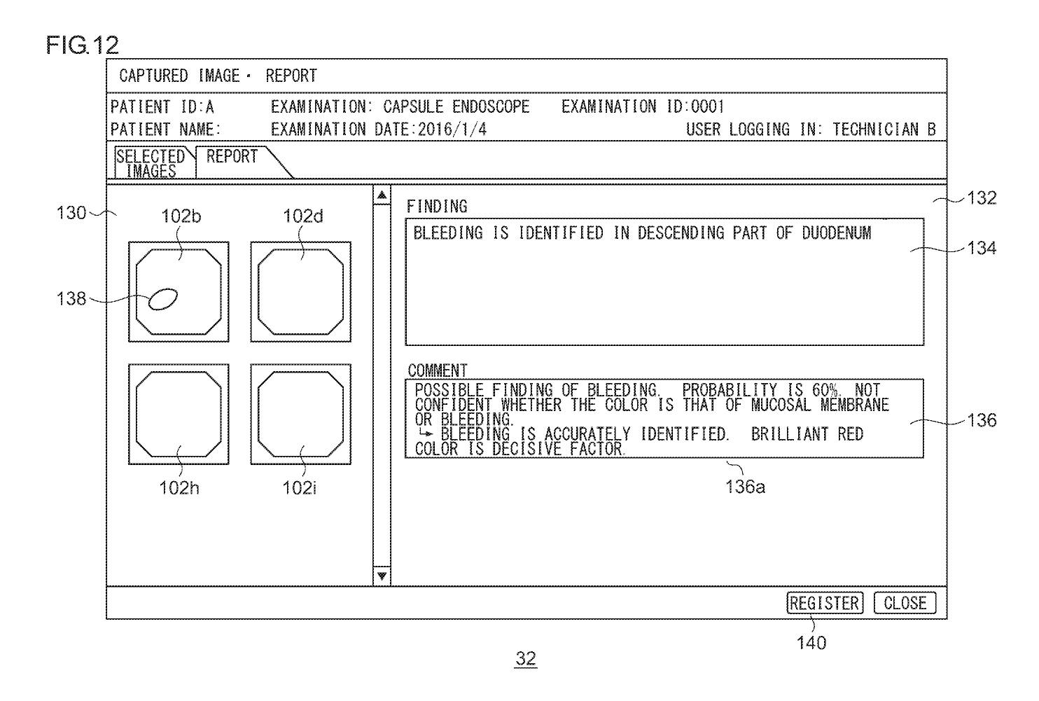

When the image interpretation by the doctor C is completed, the technician B logs into the report processing device 30 and starts the report creation application for creating an image interpretation report. FIG. 12 shows an example of the first report input screen. The coordination processing unit 58 reads the input data stored in the second image interpretation information storage unit 18 and causes the input data to be reflected in the comment input area 136 in the first report input screen. The coordination processing unit 58 reads the input data in the comment input area 158, i.e., a comment 136a by the doctor C, from the second image interpretation information storage unit 18 and causes the comment to be displayed in the comment input area 136. This allows the technician B to resolve the questions and facilitate subsequent image observation.

In the embodiment, the technician B (assistant) and the doctor C perform image interpretation independently. Therefore, the image interpretation reports are created separately. For this reason, the revisions of the image interpretation reports by the technician B and the revisions of the image interpretation reports by the doctor C are separately managed. If, for example, the doctor overwrites and updates the image interpretation report created by the technician B, the revision number of the image interpretation reports will increase as a result. In the embodiment, the doctor C creates the image interpretation reports independently of the technician B so that, advantageously, the revision number is prevented from increasing unnecessarily.

Described above is an explanation based on an exemplary embodiment. The embodiment is intended to be illustrative only and it will be understood by those skilled in the art that various modifications to constituting elements and processes could be developed and that such modifications are also within the scope of the present disclosure.

In the embodiment, all input data entered by the technician B is described as being reflected in the second report input screen in which the doctor C enters data. In a variation, the doctor C may select the input data reflected.

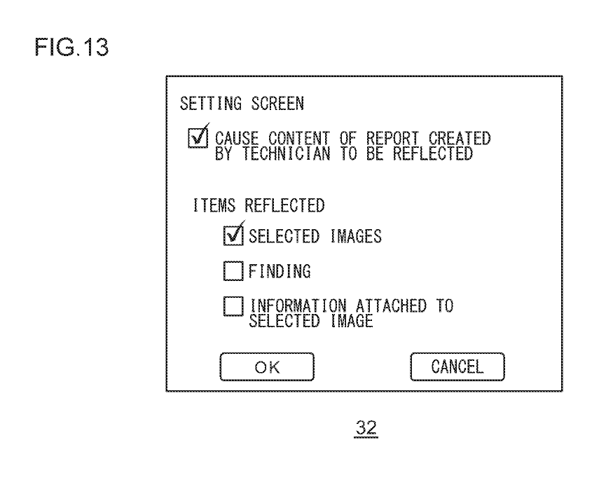

FIG. 13 shows a screen for setting items that should be reflected. The item setting unit 66 sets items that the coordination processing unit 58 reflects in the second report input screen. The item setting unit 66 causes the display device 32 to display the setting screen shown in FIG. 13 and allows the doctor C to determine items that should be reflected. The items set to be reflected may be registered in the user DB 62 for each doctor. In the example shown in FIG. 13, the checkbox for “selected image” is checked. Therefore, the coordination processing unit 58 causes only the selected image selected by the technician to be reflected in the second report input screen. By allowing setting of items that should be reflected, flexible operation is enabled.

In the embodiment, it is described that the technician B may write a question regarding the input finding, or confidence or a lack thereof in the comment input area 136 in a free format. Attribute information may be added to the comment. For example, attribute information such as “clarification required” and “question” may be added to indicate the type of comment. This allows the doctor C to understand the objective of the comment by the technician B and make a reply properly.

In the embodiment, the technician B entered a comment “The probability is 60%. Not confident” in the comment input area 136. For example, the technician B may add information indicating confidence or lack thereof to the attached image. For example, by adding the information “Confident” to the selected image 102b and adding the information “Not confident” to the selected image 102d, the doctor C can write a comment for the attached image selected by the technician B in the comment input area 158. Judgment on individual images often involves difficulty. By knowing how the technician B created the finding, the doctor C can leave a detailed comment in the comment input area 158. Accordingly, an excellent educative benefit can be expected.

It is described with reference to FIGS. 3 and 9 that the images selected by the technician B and the images selected by the doctor C are displayed in different display modes. The different display modes may be configured in further details. For example, the images selected only by the technician, the images selected only by the doctor, the images selected by the technician and canceled by the doctor, and the images selected by the technician and maintained by the doctor may be displayed in different modes.

What is claimed is:

1. An image interpretation report creation support system for supporting a task of creating an image interpretation report, comprising a processor comprising hardware, wherein the processor is configured to:
generate a first report input screen in which a first image interpreter inputs a result of image interpretation, the first report input screen not including a diagnostic input area in which to input a diagnostic result and including a first comment input area in which the first image interpreter inputs a comment for a second image interpreter;
store input data input by the first image interpreter in the first report input screen in a first storage; and
perform a coordination process of reading out, when a second report input screen in which a second image interpreter inputs a result of image interpretation and which includes a diagnostic input area in which to input a diagnostic result is generated, the input data stored in the first storage and causing the input data to be reflected in an input area in the second report input screen.

2. The image interpretation report creation support system according to claim 1, wherein the processor is configured to:
display the input data read out from the first storage in the input area in the second report input screen in a manner that it is known that the input data was entered by the first image interpreter.

3. The image interpretation report creation support system according to claim 1, wherein
the second report input screen includes a second comment input area in which the second image interpreter inputs a comment for the first image interpreter,
the processor is configured to:
store input data input by the second image interpreter in the second report input screen in a second storage; and
read out the input data stored in the second storage and cause the input data to be reflected in the first comment input area in the first report input screen.

4. The image interpretation report creation support system according to claim 3, wherein
the second storage stores the input data input in the input area in the second report input screen.

5. The image interpretation report creation support system according to claim 4, wherein
the second storage stores the input data which is stored in the first storage and reflected in the input area in the second report input screen.

6. The image interpretation report creation support system according to claim 1, wherein the processor is configured to:
set an item reflected in the second report input screen.

7. The image interpretation report creation support system according to claim 1, wherein
the image interpretation report creation support system is a system for supporting a task of creating an image interpretation report of a capsule endoscopic examination.
